(19) United States
(12) Patent Application Publication
(10) Pub. No.: US 2010/0041991 A1
(43) Pub. Date: Feb. 18, 2010

(54) HAPTIC FEEDBACK MEDICAL SCANNING METHODS AND SYSTEMS

(75) Inventor: David N. Roundhill, Woodinville, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/442,537

(22) PCT Filed: Sep. 18, 2007
(86) PCT No.: PCT/IB2007/053773

§ 371 Date: Mar. 24, 2009

Related U.S. Application Data
(60) Provisional application No. 60/826,797, filed on Sep. 25, 2006.

(51) Int. Cl. A61B 8/14

(52) U.S. Cl. 600/443; 600/459

(57) ABSTRACT

Devices for use in medical imaging can include a robotic arm (220) having multiple degrees-of-freedom movement capability, a scanning transducer (230) coupled in proximity to an end of the robotic arm, and a haptic interface (250) having one or more mechanical linkages and being in communication with the robotic arm, and adapted to issue command signals to move the robotic arm in one or more directions or angles and to receive feedback signals from the robotic arm.

### BACKGROUND

There are a variety of medical imaging technologies used in modern medicine including X-ray photography, linear-tomography, poly-tomography, Computerized Axial Tomography (CAT/CT), NucleoMagnetic Resonance (NMR) and ultrasonic imaging. Of all these technologies, only ultrasonic imaging requires the direct hands-on attention of a medical professional often referred to as a “sonographer”. For example, while technicians routinely take X-ray images of a patient from the vantage of a completely different room in order to avoid radiation exposure, a sonographer must physically hold and subtly manipulate an ultrasonic transducer against a patient's skin in order to get meaningful images.

While the known manual methods of ultrasonic imaging are generally safe and work well for most situations, there are a number of scenarios where these traditional methods pose uncomfortable or potentially dangerous situations for the sonographer. For instance, during surgery it may be necessary for a sonographer to provide constant image feedback for the surgeon, but doing so requires that the sonographer pose in highly contorted and uncomfortable positions for long periods of time—a practice that over time can result in a long-term disability of the sonographer. Also, in situations where the patient is located in a physically hazardous environment, such as in an X-ray laboratory, simultaneously taking X-ray and ultrasonic images can be both difficult and hazardous for the sonographer. Accordingly, new methods and systems relating to ultrasonic imaging are desirable.

### SUMMARY

In an illustrative embodiment, a haptic system for use in medical imaging includes a robotic arm having multiple degrees-of-freedom movement capability, a scanning transducer coupled in proximity to an end of the robotic arm, and a haptic interface having one or more mechanical linkages and being in communication with the robotic arm, and adapted to issue command signals to move the robotic arm in one or more directions or angles and to receive feedback signals from the robotic arm.

In another illustrative embodiment, haptic system configured to enable an operator to remotely perform a medical scanning procedure on a patient includes a scanning transducer having one or more force sensors coupled thereto, and a haptic control means for issuing command signals capable of controlling the position and angle of the scanning transducer relative to a patient, and for receiving feedback signals for providing tactile feedback to an operator handling the haptic control means.

In yet another illustrative embodiment, a method for enabling an operator to perform an ultrasonic medical image scan on a patient from a remote position includes generating command signals by a haptic device in response to mechanical manipulation by an operator, positioning a robotic arm having an ultrasonic transducer coupled thereto in response to the generated command signals such that the ultrasonic transducer makes physical contact with the patient, sensing at least one of position and force feedback signals from the robotic arm and causing the haptic device to conform to the feedback signals.

### DESCRIPTION OF THE DRAWINGS

The illustrative embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 depicts an illustrative block diagram of a networked medical imaging system using haptic feedback technology;

FIG. 2 depicts an exemplary ultrasonic imaging device used in conjunction with a robotic arm;

FIG. 3 depicts an exemplary ultrasonic transducer with various force vectors of interest acting upon it;

FIG. 4 depicts an exemplary haptic controller;

FIG. 5 is a block diagram of an exemplary control system useable with a hapticly controlled imaging system;

FIG. 6 is an exemplary control model for use with a hapticly controlled ultrasonic imaging system; and

FIG. 7 is a block diagram outlining various exemplary operations directed to the haptic control of a medical imaging device.

### DETAILED DESCRIPTION

In the following detailed description, for purposes of explanation and not limitation, illustrative embodiments disclosing specific details are set forth in order to provide a thorough understanding of an embodiment according to the present teachings. However, it will be apparent to one having ordinary skill in the art having had the benefit of the present disclosure that other embodiments according to the present teachings that depart from the specific details disclosed herein remain within the scope of the appended claims. Moreover, descriptions of well-known apparatus and methods may be omitted so as to not obscure the description of the illustrative embodiments. Such methods and apparatus are clearly within the scope of the present teachings.

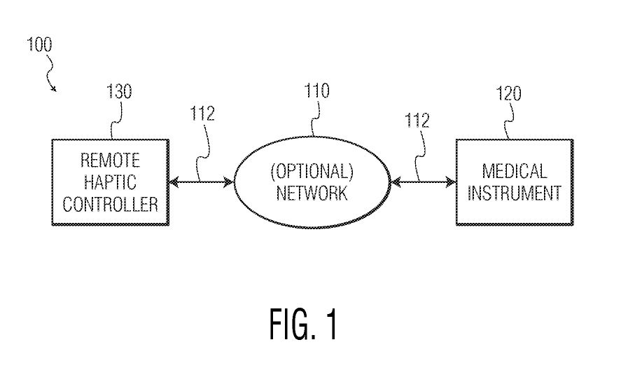

FIG. 1 depicts an illustrative embodiment of a medical imaging system 100 using haptic feedback technology. As shown in FIG. 1, the medical imaging system 100 includes a remote haptic controller 130 and a medical instrument 120 connected to a common network 110 via links 112.

In operation, an operator/sonographer located at the haptic controller 130 can manipulate a specially-configured control mechanism in order to define the spatial and angular positions of a hand-held “reference wand”. In various embodiments, the haptic controller 130 can be used to define 6 degrees-of-freedom (DOF) including the X, Y and Z positions of the reference wand (relative to some reference point) as well as the X, Y and Z angles at which the reference wand is positioned. Note that the position and angle of the reference wand can be used to define the spatial position and angle of an ultrasonic transducer (relative to a patient) located at the medical instrument 120.

While the exemplary haptic controller 130 is a 6-DOF system, in other embodiments a 7-DOF haptic controller can be used that further includes a rotational degree of freedom about the central-axis of the reference wand thus allowing the sonographer to spin the wand (and by default an ultrasonic transducer) on its central-axis. In other embodiments, however, fewer than six degrees of freedom can be used. For example, in one embodiment a 4-DOF system using a single linear direction control and three dimensional angular control can be used, while in other embodiments a 1-DOF system capable of being manipulated along a single linear direction may be used. Notably, there are comparatively few cases where rotation would be required.

During operation, as the sonographer manipulates the haptic controller's reference wand, the exemplary haptic controller 130 can send some form of control signals representing the position and angles of the reference wand, and/or control signals representing the forces that the sonographer applies to the reference wand, to the medical instrument 120 via the network 110 and links 112.

In turn, a robotic arm carrying the aforementioned ultrasonic transducer at the medical instrument 120 can react to the control signals, i.e., change the position and angle of the ultrasonic transducer in a manner that would be consistent/conform with the position and angles of the haptic controller's reference wand—or otherwise mimic those forces that the sonographer applies to the reference wand.

As the robotic arm reacts to conform with the control signals, various position and force sensors located in the robotic arm and/or coupled to the ultrasonic transducer can provide various feedback signals to the haptic controller 130. For example, by coupling one or more force sensors to the ultrasonic transducer to detect forces applied to the transducer, the medical instrument 120 can provide feedback signals to the haptic controller 130 that can be used to create analogous forces against the hand of the sonographer to effectively simulate the tactile feel that the sonographer would experience as if he were directly manipulating the transducer at the medical instrument 120.

In addition to a haptic interface, the haptic-controller 130 and medical instrument 120 can optionally include some form of system to remotely control the “back end” of the ultrasonic instrumentation supporting the ultrasonic transducer. For example, by providing a personal computer at the haptic controller 130 containing a specially designed software package, the sonographer can change any number of the ultrasonic instrument's settings, such as its frequency and power settings, that the sonographer would otherwise need direct access to the ultrasonic instrument's front panel. Additionally, any image that might be generated at the ultrasonic instrument's display can be optionally sent to the personal computer for more convenient display to the sonographer.

The illustrative network 110 is an Ethernet communication system capable of passing IEEE1588 compliant signals. However, in other embodiments the network 110 can be any viable combination of devices and systems capable of linking computer-based systems. The network 110 may include, but is not limited to: a wide area network (WAN), a local area network (LAN), a connection over an intranet or extranet, a connection over any number of distributed processing networks or systems, a virtual private network, the Internet, a private network, a public network, a value-added network, an Ethernet-based system, a Token Ring, a Fiber Distributed Datalink Interface (FDDI), an Asynchronous Transfer Mode (ATM) based system, a telephony-based system including T1 and E1 devices, a wired system, an optical system, or a wireless system. Known protocols for each of the noted networks are included and are not detailed here.

The various links 112 of the present embodiment are a combination of devices and software/firmware configured to couple computer-based systems to an Ethernet-based network. However, it should be appreciated that, in differing embodiments, the links 112 can take the forms of Ethernet links, modems, networks interface card, serial buses, parallel busses, WAN or LAN interfaces, wireless or optical interfaces and the like as may be desired or otherwise dictated by design choice.

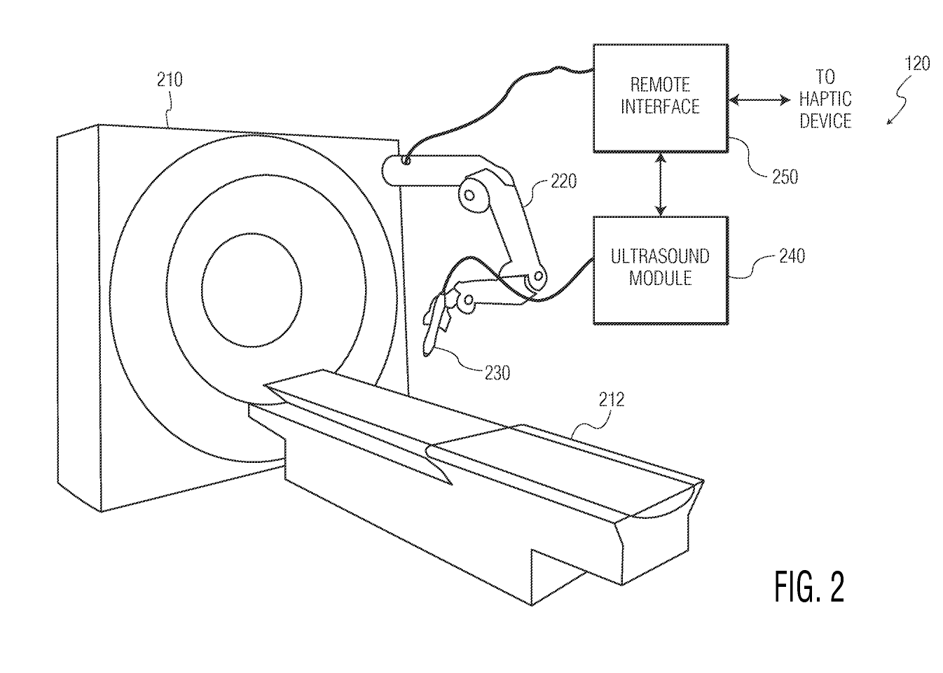

FIG. 2 depicts an ultrasonic imaging system 120 used in conjunction a CT scanning system 210 in accordance with an illustrative embodiment. As shown in FIG. 2, the CT scanning system 210 is accompanied by a bed 212 upon which a patient might rest. A 6-DOF robotic arm 220 is attached to the CT scanning system 210, and an ultrasonic transducer 230 is coupled at the end of the robotic arm 220. A remote interface 250 is further coupled to the robotic arm 220, and a back-end ultrasonic module 240 is coupled to the ultrasonic transducer 230. Notably, the bed 212 may be any structure adapted to translate a patient through the CT scanning system 210. Also, it may be useful to couple the translation of the bed 212 to the control robotic arm thereby allowing the arm to move in ‘lock-step’ with the bed 212.

In operation, control signals sent by an external device, such as a haptic controller, can be received by the remote interface 250. The remote interface 250 can condition, e.g., scale, the received control signals and forward the conditioned control signals to the robotic arm 220. In turn, the robotic arm 220 can change the position and angle of the transducer 230 to conform with the conditioned control signals.

As the robotic arm reacts to conform with the control signals, various position sensors within the robotic arm (not shown) and force sensors coupled to the transducer (also not shown) can be used to provide tactile feedback to a remotely positioned sonographer using a haptic controller via the remote interface 250. For example, assuming that the robotic arm 220 positions the face of the transducer 230 against a patient's abdomen, the force sensors can detect the forces between the transducer 230 and the patient. The detected forces, in turn, can be used to generate an analogous set of forces against the sonographer's hand using a haptic controller. Accordingly, the sonographer can benefit from an extremely accurate tactile feel without needing to be exposed to any radiation produced by the CT device 210.

As the ultrasonic transducer 230 is advantageously positioned against a patient, the ultrasound module 240 can receive those ultrasonic reflection signals sensed by the ultrasonic transducer 230, generate the appropriate images using a local display and/or optionally provide any available image to the sonographer via the remote interface 250. Additionally, the sonographer can change various settings of the ultrasound module 240 via the remote interface 250 as would any sonographer in the direct presence of such an ultrasonic imaging instrument.

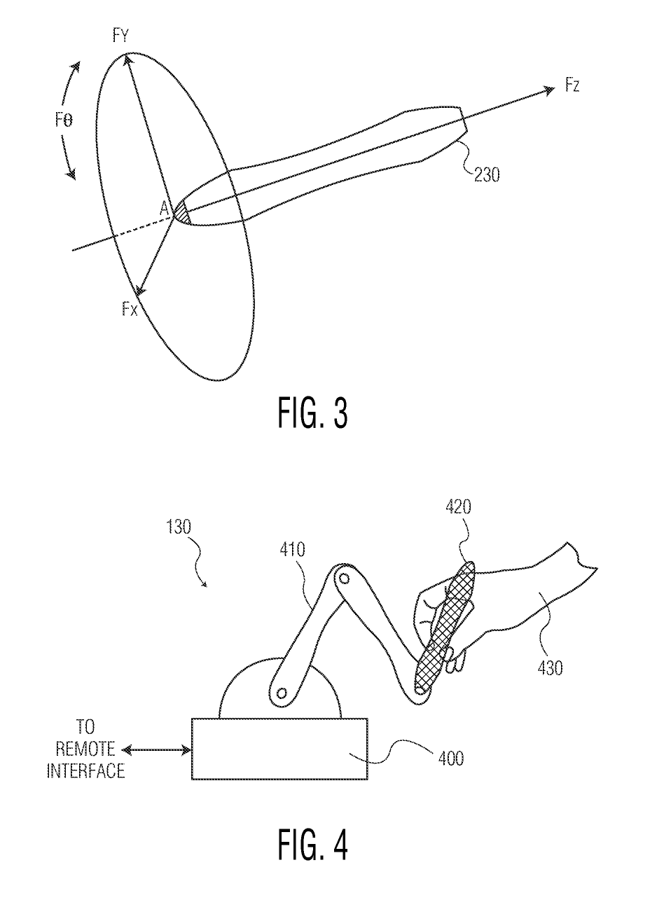

FIG. 3 depicts the ultrasonic transducer 230 of FIG. 2 along with various force vectors of interest that may be used to provide tactile feedback to a sonographer. As shown in FIG. 3 the ultrasonic transducer 230 has a central axis running along the length of the ultrasonic transducer 230 upon which a first force vector F_{Z} representing a force applied against the front tip/face (at point A) of the ultrasonic transducer 230 is shown.

In addition to the force vector F_{Z} along the central axis, it can be advantageous to measure forces applied laterally to the transducer's front face, such as those represented by force vectors F_{X} and F_{Y} that can exist in a plane normal to force vector F_{Z} and normal to one another. Sensing forces along vectors F_{X} and F_{Y} can provide an enhanced tactile feedback to the sonographer, such as the tactile feel of the friction and pressure that occur when a transducer's face is dragged along the surface of a patient's skin.

Still further, in order to provide tactile feedback in situations where a sonographer might wish to rotate the transducer 230 while in contact with a patient's skin, a rotational force about the central axis of the transducer 230, represented by force vector F_{θ}, can be optionally detected.

Continuing to FIG. 4, a haptic controller 130 of an illustrative embodiment is shown. The haptic controller 130 includes a base 400 having a mechanical armature/linkage 410 onto which a reference wand 420 is appended. The exemplary reference wand 420 is shaped like the transducer 230 of FIGS. 2 and 3, but of course the particular configuration of the reference wand 420 can change from embodiment to embodiment.

The haptic controller 130 of the illustrative can be configured to sense the position of the tip of the reference wand 420 in three dimensions, as well as the angle of the reference wand 420 in three dimensions, relative to the base 400 using a number of position sensors (not shown). In some embodiments, the reference wand 420 can additionally be equipped to sense a rotation (or rotational force) about the central axis of the reference wand, while in other embodiments the haptic controller 130 as a whole may have less than 6 degrees-of-freedom.

Further, in order for the haptic device 130 to provide an appropriate tactile feedback to a sonographer's hand 430, a number of force sensors and drive motors (not shown) can be installed. Thus, when the proper controls and interfaces are applied to the haptic device 130 and a respective robotic arm and transducer, any force applied to the reference wand 420 by the sonographer's hand 430 can be countered by tactile feedback provided by the respective robotic arm and transducer.

Examples of various haptic controllers useable for some embodiments include the PHAMTOM® Omni device, the PHAMTOM® Desktop device, the PHAMTOM® Premium device, and the PHAMTOM® Premium 6DOF device made by SensAble Technologies, Inc. located at 15 Constitution Way, Woburn, Mass.

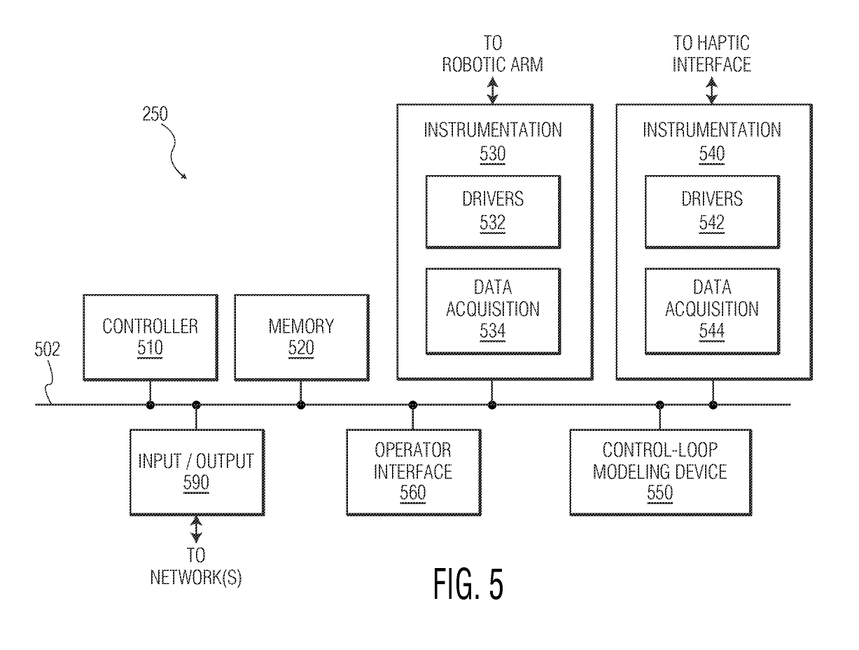

FIG. 5 is a block diagram of a remote interface 250 of an illustrative embodiment that is adapted for use with a haptic controlled imaging system. The remote interface 250 can include a controller 510, a memory 520, a first set of instrumentation 530 having a first set of drivers 532 and first data acquisition device 534, a second set of instrumentation 540 having a second set of drivers 542 and second data acquisition device 544, a control-loop modeling device 550, an operator interface 560 and an input/output device 590. The controller 510 does not necessarily, mimic the coarse movements of the robotic arm, but rather the pressure applied by the robotic arm in 3D space. If there is no resistance (i.e. no force) applied in response to force applied by the controller, a coarse motion of the robotic arm results in response to the force applied to the controller.

Although the remote interface 250 of FIG. 5 uses a bussed architecture, many other architectures contemplated for use as would be appreciated by one of ordinary skill in the art. For example, in various embodiments, the various components 510-590 can take the form of separate electronic components coupled together via a series of separate busses or a collection of dedicated logic arranged in a highly specialized architecture.

It also should be appreciated that portions or all of some of the above-listed components 530-590 can take the form of software/firmware routines residing in memory 520 and be capable of being executed by the controller 510, or even software/firmware routines residing in separate memories in separate servers/computers being executed by different controllers.

In operation, the remote interface 250 can receive control signals from a haptic controller, such as that shown in FIG. 4, via the second data acquisition device 544, then process the control signals using the control-loop modeling device 550. Various processing for the received control signals can include changing the gain of the control signals to increase or decrease sensitivity, adding a governor/limiter on the control signals to limit a maximum position or force that the respective robotic arm should be capable of exhibiting and so on. In an embodiment, a “deadman” safety is provided to the robotic arm via the control signals. Such a feature is useful, for example, if the network communication link is disrupted, the applied pressure is zeroed.

Once the control signals have been conditioned, the control signals can be fed to the respective robotic arm (via drivers 532) while bring further processed according to a complex control loop in the control-loop modeling device 550 using optional feed-forward and feedback compensation.

Simultaneously, the first data acquisition device 534 can receive position and/or force feedback information from the respective robotic arm, and optionally condition the feedback information in much the same way as the control information, e.g., by changing gain or imposing a more complex transfer function. The conditioned feedback information can then be provided to the haptic controller (via drivers 542) while being processed according to the control loop processes modeled in the control-loop modeling device 550.

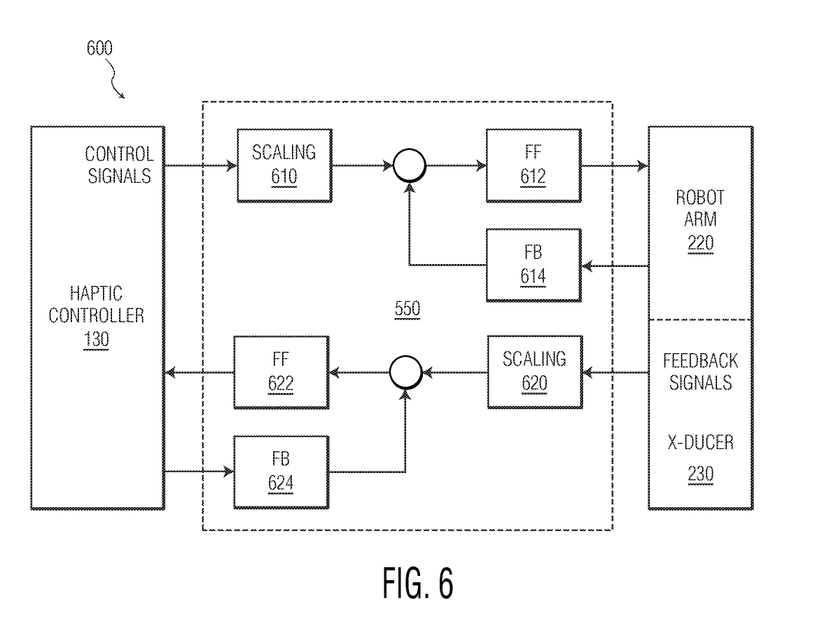

FIG. 6 depicts a control model 600 for use with a haptic controlled imaging system in accordance with an illustrative embodiment. As shown in FIG. 6, a first scaling module 610 can receive control signals, typically position or force data, from a haptic controller 130 where it can then be processed according to a control loop involving a first feed-forward compensation module 612, the mechanics of the robot arm 220 and a first feedback compensation module 614.

Similarly, a second scaling module 620 can receive position and/or force feedback signals from the robotic arm 220 and transducer 230 where the feedback signals can then be processed according to a second control loop involving a second feed-forward compensation module 622, the mechanics of the haptic controller 130 and a second feedback compensation module 624.

Note that when the control signals provided by the haptic controller 130 primarily consist of position information, the subsequent (upper) control loop will be a position control loop, the feedback signals will primarily consist of force information and the subsequent (lower) control loop will be a force control loop. Conversely, when the control signals provided by the haptic controller 130 primarily consist of force information, the upper control loop will be a force control loop, the feedback signals will primarily consist of position information and the lower control loop will be a position control loop.

Also note that the particular control model portrayed in FIG. 6 is purely exemplary, and practical control models should not be limited to the sole embodiment illustrated of FIG. 6.

Returning to FIG. 5, as the various instrumentation 530 and 540 and control-loop modeling device 550 enable a sonographer to remotely position an ultrasonic transducer with tactile feedback, the operator interface 560 and input/output device 590 optionally can be used to remotely configure the back-end of the ultrasonic instrumentation connected to an ultrasonic transducer in much the same fashion as a sonographer having hands-on access might do. Additionally, the operator interface 560 and input/output device 590 may be used to convey ultrasonic image data from the ultrasonic instrumentation to the sonographer.

Note that in various embodiments the remote interface 250 can be divided into two or more portions, which may be advantageous when a haptic control device and a robotic arm are separated by appreciable distances. For example, two separate interfaces 250A and 250B might be used with remote interface 250A located by a haptic controller and remote interface 250B located by the respective robotic arm. In this example, remote interface 250A can drive the servo-mechanisms and collect transducer data of the haptic controller, and remote interface 250B can drive the servo-mechanisms and collect transducer data of the robotic arm and ultrasonic transducer. Control and feedback data can be exchanged via the respective input/output devices, and overall control may be delegated to one of the two remote interfaces 250A and 250B.

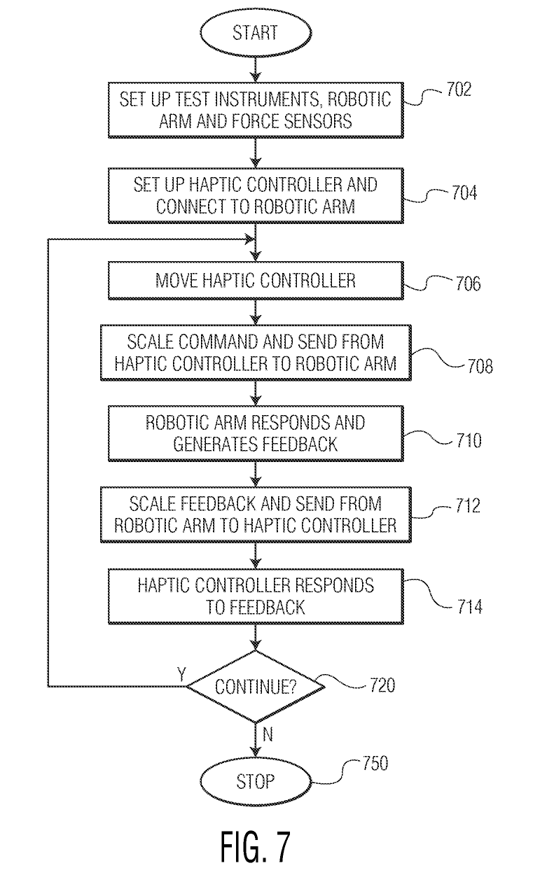

FIG. 7 is a block diagram outlining various exemplary operations directed to the haptic control of a medical imaging device. The process starts in step 702 where an ultrasonic imaging instrument (or similarly situated medical device) is set up along with a robotic arm coupled to the ultrasonic imaging instrument's transducer plus a number of force sensors. Next, in step 704, a haptic controller is similarly set up and communicatively connected to the robotic arm and transducer of step 702. Control continues to step 706.

In step 706, an operator, such as a trained sonographer, can move a control surface (e.g., a reference wand) of the haptic controller to generate force or position control signals. Next, in step 708, the control signals can be optionally scaled or otherwise processed, and then sent to the robotic arm of step 702. Control continues to step 710.

In step 710, the robotic arm can react to the scaled/processed control signals, and during the reaction process generate position and/or force feedback signals. Next, in step 712, the feedback signals can be optionally scaled/processed and then sent to the haptic controller. Then, in step 714, the haptic controller can respond to the feedback signals to give the sonographer a tactile feel of the ultrasonic transducer. Control continues to step 720.

In step 720, a determination is made as to whether to continue to operate the controlled haptic feedback process described in steps 706-714. If the haptic feedback processes to continue, control jumps back to step 706; otherwise, control continues to step 750 where the process stops.

In various embodiments where the above-described systems and/or methods are implemented using a programmable device, such as a computer-based system or programmable logic, it should be appreciated that the above-described systems and methods can be implemented using any of various known or later developed programming languages, such as “C”, “C++”, “FORTRAN”, Pascal”, “VHDL” and the like.

Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform the above-described systems and/or methods.

For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to effect these techniques, while remaining within the scope of the appended claims.

1. A haptic system (100) for use in medical imaging, the system comprising:
a robotic arm (220) having multiple degrees-of-freedom movement capability;
a scanning transducer (230) coupled in proximity to an end of the robotic arm; and
a haptic interface (250) having one or more mechanical linkages and being in communication with the robotic arm, and adapted to issue command signals to move the robotic arm in one or more directions or angles and to receive feedback signals from the robotic arm.

2. The haptic system of claim 1, wherein the scanning transducer is an ultrasonic transducer capable of providing ultrasonic image data to an ultrasonic imaging system (120).

3. The haptic system of claim 1, further comprising one or more force sensors coupled to the scanning transducer.

4. The haptic system of claim 3, wherein the one or more force sensors includes a first force sensor capable of sensing a force along a central axis of the scanning transducer.

5. The haptic system of claim 4, wherein the one or more force sensors further includes one or more second force sensors capable of sensing a lateral force against the scanning transducer, the lateral force being in a plane normal to the central axis of the scanning transducer.

6. The haptic system of claim 4, wherein the one or more force sensors further includes one or more second force sensors capable of sensing a rotational force about the central axis of the scanning transducer.

7. The haptic system of claim 4, wherein the haptic interface is capable of receiving force-related feedback signals derived from the one or more force sensors, and wherein the haptic interface is capable of exhibiting a force consistent with the force-related feedback signals against a hand of an operator in contact with the haptic interface.

8. The haptic system of claim 1, wherein the robotic arm is at least a 3 degrees-of-freedom device, the degrees-of-freedom being selected from the following: an x-position of the scanning transducer, a y-position of the scanning transducer, a z-position of the scanning transducer, an x-angle of the scanning transducer, a y-angle of the scanning transducer, a z-angle of the scanning transducer and an angle of axial rotation of the scanning transducer.

9. The haptic system of claim 1, wherein the robotic arm is a 6 degrees-of-freedom device, the degrees-of-freedom being selected from the following: an x-position of the scanning transducer, a y-position of the scanning transducer, a z-position of the scanning transducer, an x-angle of the scanning transducer, a y-angle of the scanning transducer, a z-angle of the scanning transducer and an angle of axial rotation of the scanning transducer.

10. The haptic system of claim 9, wherein the robotic arm is a 7 degrees-of-freedom device, the degrees-of-freedom including an x-position of the scanning transducer, a y-position of the scanning transducer, a z-position of the scanning transducer, an x-angle of the scanning transducer, a y-angle of the scanning transducer, a z-angle of the scanning transducer and an angle of axial rotation of the scanning transducer.

11. The haptic system of claim 1, wherein the robotic arm is configured to receive position command signals from the haptic interface and further configured to conform with the received position command signals.

12. The haptic system of claim 11, wherein the haptic interface is configured to receive force feedback signals from the robot arm, and further configured to conform with the received force feedback signals.

13. The haptic system of claim 1, wherein the robotic arm is configured to receive force command signals from the haptic interface, and further configured to conform with the received force command signals, and wherein the haptic interface is configured to receive position feedback signals from the robot arm, and further configured to conform with the received position feedback signals.

14. The haptic system of claim 4, wherein at least one of a force command signal and a sensed force feedback signal is scaled using a non-unity transfer function in order to either increase or decrease force sensitivity of the haptic interface.

15. A haptic system configured to enable an operator to remotely perform a medical scanning procedure on a patient, the system comprising:
a scanning transducer (230) having one or more force sensors coupled thereto; and
a haptic control means (130) for issuing command signals capable of controlling the position and angle of the scanning transducer relative to a patient, and for receiving feedback signals for providing tactile feedback to an operator handling the haptic control means.

16. The haptic system of claim 15, further comprising a movement means for receiving the command signals and for changing the position and angle of the scanning transducer in response to the received the command signals.

17. A method for enabling an operator to perform an ultrasonic medical image scan on a patient from a remote position, the method comprising:
generating command signals by a haptic device in response to mechanical manipulation by an operator;
positioning a robotic arm having an ultrasonic transducer coupled thereto in response to the generated command signals such that the ultrasonic transducer makes physical contact with the patient;
sensing at least one of a position and a force feedback signals from the robotic arm; and
causing the haptic device to conform to the feedback signals.

18. The method of claim 17, wherein the robotic arm includes a number of force sensors capable of sensing one or more force vectors applied to the scanning transducer.

19. The method of claim 17, wherein the step of positioning a robotic arm is performed using a sensed force signal that is scaled using a non-unity transfer function in order to either increase or decrease force sensitivity of the haptic interface.

20. The method of claim 17, further comprising using a remote operator interface to remotely control the operational configuration of an ultrasonic imaging system coupled to the ultrasonic transducer.
